(19) United States
(12) Patent Application Publication
(10) Pub. No.: US 2025/0018396 A1
(43) Pub. Date: Jan. 16, 2025

(54) MATERIALS FOR HANDLING AND LONG-TERM STORAGE OF EXTRACELLULAR VESICLES

(71) Applicant: CORNING INCORPORATED, CORNING, NY (US)

(72) Inventors: Amy Claire Kauffman, Kittery, ME (US); Ana Maria del Pilar Pardo, Portsmouth, NH (US)

(21) Appl. No.: 18/712,121

(22) PCT Filed: Nov. 8, 2022
(86) PCT No.: PCT/US2022/049266

§ 371 Date: May 21, 2024

Related U.S. Application Data
(60) Provisional application No. 63/282,433, filed on Nov. 23, 2021.

(51) Int. Cl. B01L 3/00

(52) U.S. Cl. CPC B01L 3/508; B01L 3/505; B01L 2200/0689; B01L 2200/16; B01L 2300/042

(57) ABSTRACT

A storage container for extracellular vesicles (EVs) comprises a container and a lid or cap. The container comprises a housing having an opening or aperture; an interior volume defined within the housing; and an interior surface of the housing comprising a protein non-adherent coating. The lid or cap is configured to removably attach to the housing to seal the opening or aperture of the container. A method of storing extracellular vesicles comprises collecting EVs in a storage container; adding a buffer to the container, wherein the buffer comprises a solution comprising an enzyme and a sugar; and storing the EVs in the buffer.

This application claims the benefit of priority under 35 U.S.C. § 119 of U.S. Provisional Application Ser. No. 63/282,433 filed on Nov. 23, 2021, the content of which is relied upon and incorporated herein by reference in its entirety.

### TECHNICAL FIELD

The present disclosure generally relates to extracellular vesicles and materials for handling and storing extracellular vesicles.

### BACKGROUND

“Extracellular vesicles” or “EVs” refer to a population of particles naturally released from cells. EVs are involved with intercellular communication and are involved in many processes in health and disease states such as stress compensation, physiological responses, homeostasis, and various other biological regulatory activities. Because of their therapeutic potential in providing the necessary factors to mediate physiological events, as well as their ability to serve as less invasive diagnostic markers for prognosis of pathological conditions, EVs continue to be of interest to the scientific and medical communities.

However, as with many biological and acellular products, there is significant challenge in producing, handling, and collecting these biologic products. Many of these biomolecules or vesicles are shrouded in proteins, lipids, and other components that make them susceptible to sticking on a variety of surfaces or to each other. This is particularly evident when handling EVs with pipettes, centrifuge tubes, or other plastic surfaces. Significant adsorption of EVs occurs during handling processes, which leads to a significant reduction in total EV yield. A need exists for materials that may reduce the drawbacks associated with handling EVs.

### SUMMARY

In an aspect, a storage container for extracellular vesicles (EVs) is provided. The storage container comprises a container comprising a housing having an opening or aperture; an interior volume defined within the housing; and an interior surface of the housing comprising a neutral, hydrophilic polymer coating that reduces binding of attachment proteins; and a lid or cap configured to removably attach to the housing to seal the opening or aperture of the container. In an embodiment, the housing comprises a top, a bottom, and one or more sidewalls.

In an embodiment, the storage container is configured to store EVs up to 7 days. In an embodiment, the storage container is configured to store EVs for up to 30 days. In an embodiment, the storage container is configured to store EVs for 7 days to 30 days.

In an embodiment, the EV storage container is configured to store EVs without loss in yield. In an embodiment, the EV storage container is configured to store EVs without sample contamination. In an embodiment, the EV storage container is configured to store EVs without leachable contamination from the storage container.

In an embodiment, the EV storage container is configured to store EVs at a temperature from 25° C. to −80° C.

In an embodiment, the storage container comprises a tube, a vial, or a flexible bag. In an embodiment, the tube comprises a centrifugation tube, conical tube, or a culture tube.

In an embodiment, an interior surface of the lid or cap is configured to be in contact with the interior volume of the container when the lid or cap is attached to the housing. In an embodiment, the interior surface of the lid or cap comprises a neutral, hydrophilic polymer coating that reduces binding of attachment proteins.

In an embodiment, the lid or cap comprises threads compatible with and configured to interlocking with threads on an exterior of the housing to seal the opening or aperture of the container.

In an embodiment, the lid or cap is configured to snap on an exterior of the housing to seal the opening or aperture of the container.

In an aspect, a method of storing extracellular vesicles (EVs) is provided. The method comprises collecting EVs in a storage container according to embodiments of the present disclosure. The method further comprises adding a buffer to the container, wherein the buffer comprises a solution comprising an enzyme and a sugar. The method further comprises storing the EVs in the buffer.

In an embodiment, the enzyme may comprise endonucleases, exonucleases, DNAses, RNases, strand-specific nucleases, Cas9 or other CRISPR associated protein nucleases, or combinations thereof. Nonlimiting examples of enzymes include DECONTAMINASE (a binuclease endonuclease available from AG Scientific Inc., San Diego, CA), recombinant Dr. Nuclease (a recombinant endonuclease available from Syd Labs, Boston, MA), PIERCE Universal Nuclease (a nuclease available from Thermo Fisher Scientific Inc., Waltham, MA), BENZALT (a genetically engineered non-specific endonuclease available from BioVision Inc., Milpitas, CA), CYANASE (a non-specific endonuclease available from Biophoretics, Sparks, NV), and BENZONASE (a genetically engineered endonuclease available from Merck KGaA, Darmstadt, Germany).

In an embodiment, the sugar may comprise cellobiose, chitobiose, isomaltose, kestose, lactose, lactulose, maltose, maltotriose, maltotriulose, mannobiose, melezitose, melibiose, nigerotriose, raffinose, sophrose, sucrose, trehalose, turanose, xylobiose, or combinations thereof.

In an embodiment, storing the EVs in buffer comprises storage for 7 days to 30 days. In an embodiment, storing the EVs in buffer comprises storage for up to 30 days. In an embodiment, storing the EVs in buffer comprises storage for up to 7 days. In an embodiment, storing the EVs in buffer comprises storage for up to 7 days without loss in EV yield.

In an embodiment, storing the EVs in buffer comprises storage at temperatures in a range of about 25° C. to about −80° C. In an embodiment, storing the EVs in buffer comprises storage at 4° C. for up to 5 days.

In an embodiment, collecting EVs in a container further comprises collecting EVs post-diafiltration.

In an embodiment, a freeze-thaw cycle comprises thawing the buffer comprising EVs from a frozen temperature to a room temperature. In an embodiment, storing the EVs in buffer comprises storage over one or more freeze-thaw cycles. In an embodiment, storing the EVs in buffer comprises storage for up to three freeze-thaw cycles.

Additional aspects of the present disclosure will be set forth, in part, in the following detailed description, figures, and claims, and in part will be derived from the detailed description, or can be learned by practice of the disclosure. It is to be understood that both the foregoing general description and the following detailed description are exemplary and explanatory only and are not restrictive of the disclosure as disclosed.

### BRIEF DESCRIPTION OF THE DRAWINGS

FIG. 1A shows a side view of a storage container according to an embodiment of the present disclosure.

FIG. 1B shows a cross-sectional side view of a storage container according to an embodiment of the present disclosure.

FIG. 2 shows a graphical image depicting the concentration of EVs over time in embodiments of storage containers according to the present disclosure.

FIG. 3 shows a graphical image depicting the diameter of EVs over time in embodiments of storage containers according to the present disclosure.

FIG. 4 shows a graphical image depicting the concentration of EVs over freeze-thaw cycles in embodiments of storage containers according to the present disclosure.

FIG. 5 shows a graphical image depicting the diameter of EVs over freeze-thaw cycles in embodiments of storage containers according to the present disclosure.

### DETAILED DESCRIPTION

Materials for storage and handling of extracellular vesicles (EVs), the nanometer-scale materials that stick or bind to plastic surfaces during use, are described herein that may reduce the drawbacks associated with handling EVs. In an embodiment, coated vessels, liquid transfer devices (such as pipette tips), and/or containers are described that may be used for storage and handling of extracellular vesicles. In an embodiment, a formulated buffer is described herein that may prevent extracellular vesicle aggregation and ablation during handling and long-term cold storage.

To reduce total EV loss, conventional techniques include coating conventional tubes with protein such Bovine Serum Albumin (BSA) or using Eppendorf Protein LoBind tubes (Eppendorf S E, Hamburg, Germany). However, BSA coating is not a permanent coating and coating tubes with protein presents a significant problem because resulting EV samples become contaminated with the introduced foreign proteins, thereby leading to undesirable downstream cell culture or misleading characterization results.

As described herein, storage containers are provided for storage and handling of EVs. A buffer is also described herein for storage and handling of EVs. Use of the storage containers and buffer as described herein provides a dual approach to prevent EV adsorption to plastic surfaces as well as self-aggregation of EVs. Such an approach offers technical advantages to address both pathways for EV loss and/or destruction and does not introduce any contaminants, such as proteins or other synthetic molecules, in contrast to other conventionally-used coatings or buffers used in the field.

In an aspect, a storage container for extracellular vesicles (EVs) is provided. The storage container may comprise any suitable container that allows for handling or storage of biological materials. The storage container comprises a container and a lid or cap. The container comprises a housing having an opening or aperture; an interior volume defined within the housing; and an interior surface of the housing comprising a protein non-adherent coating. The lid or cap is configured to removably attach to the housing to seal the opening or aperture of the container.

In an aspect, storage containers described herein prevent adsorption of EVs to the handling vessels or containers. In an aspect, an interior surface of the storage containers described herein may comprise a neutral, hydrophilic polymer coating that reduces binding of attachment proteins. In an embodiment, the neutral, hydrophilic polymer coating that reduces binding of attachment proteins may comprise a proprietary Ultra Low Attachment (ULA) coating. A Ultra-Low Attachment (ULA) coating is a covalently bonded coating that is hydrophilic, biologically inert and non-degradable. In an embodiment, a proprietary Ultra Low Attachment (ULA) coating may be applied to a centrifuge tube for handling and storage of EVs. The ULA coating serves to prevent adsorption and binding of EVs on cell culture flasks, plates, and vessels. In an embodiment, the storage container may comprise a ULA coated tube, vessel, or other container.

In an embodiment, the storage container may be configured to store EVs up to 7 days. In an embodiment, the storage container may be configured to store EVs up to 3 freeze-thaw cycles. In an embodiment, the EV storage container may be configured to store EVs at a temperature from 25° C. to −80° C. In an embodiment, the EV storage container may be configured to store EVs without degradation or lowering of total EV concentration. In an embodiment, the storage container may be configured to store EVs for at least 7 days. In an embodiment, the storage container may be configured to store EVs for 7 days to 30 days. In an embodiment, the storage container may be configured to store EVs up to 30 days.

In an embodiment, the storage container comprises a flexible storage container. A nonlimiting example of a flexible storage container is a flexible bag, such as a flexible media bag or a flexible bag suitable for cell culture, intravenous medications, and biologics. Such a flexible bag may be used for clinical applications.

In an embodiment, the storage container comprises a rigid storage container. In an embodiment, the storage container comprises a tube or vial. In an embodiment, the storage container comprises a centrifugation tube. In an embodiment, the storage container comprises a cylindrical vial with a removable, push-on lid or screw-on lid.

In an embodiment, an interior surface of the lid or cap is configured to be in contact with the interior volume of the container when the lid or cap is attached to the housing. In an embodiment, the interior surface of the lid or cap comprises a protein non-adherent coating. In an embodiment, the lid or cap comprises threads compatible with and configured to interlocking with threads on an exterior of the housing to seal the opening or aperture of the container.

In an embodiment, the housing comprises a top, a bottom, and one or more sidewalls. Non-limiting examples of storage containers comprising a top, a bottom, and one or more sidewalls include a flask such as a cell culture flask, multi-layer cell culture flask, or microcavity cell culture flask and having a threaded cap that screws on to an opening on the flask, a cell culture dish such as a petri dish with attachable lid that presses on to attach to the petri dish, a cell culture vessel or multi-layer cell culture vessel such as a CellSTACK (Corning Incorporated, Corning, NY), CellCube (Corning Incorporated, Corning, NY), or HYPERStack (Corning Incorporated, Corning, NY), a bioreactor such as an ASCENT fixed bed bioreactor (Corning Incorporated, Corning, NY), a cell culture plate with attachable, removable lid, and a multi-well cell culture plate with attachable, removable lid or microcavity cell culture plate with attachable, removable lid.

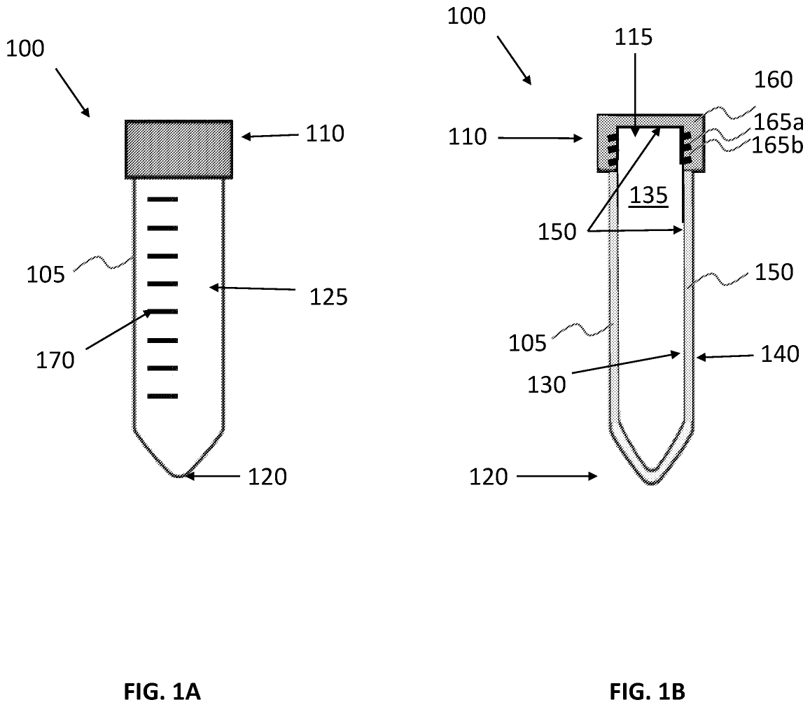

FIG. 1A shows a side view of a storage container 100 according to an embodiment of the present disclosure, while FIG. 1B shows a cross-sectional side view of storage container 100. The storage container 100 is configured for storing extracellular vesicles (EVs) according to an embodiment of the present disclosure. The storage container 100 comprises a container, such as a conical tube. The container comprises a housing 105. The housing 105 has a top 110, a bottom 120, and one or more sidewalls 125 disposed between the top 110 and bottom 120. The housing 105 has an interior surface 130 and an exterior surface 140. The housing 105 has an opening or aperture 115, such as at a top 110 of the housing 105.

An interior volume 135 is defined within the housing 105. The housing 105 defines interior volume 140 that is in contact with an interior surface 130 of the housing 105. A coating 150 is applied on the interior surface 130. The coating 150 on the interior surface 130 of the housing may comprise a neutral, hydrophilic polymer coating that reduces binding of attachment proteins.

The storage container 100 further comprises a lid or cap 160. The lid or cap 160 is configured to removably attach to the housing 105 to seal the container 100. For example, the lid 160 may attach to the housing 105 to seal the opening or aperture 115 of the container 100. A coating 150 may be applied on the interior surface of the lid 160, which may be in contact with contents of the container 100 stored within the interior volume 135. The coating 150 on the interior surface of the lid 160 may comprise a neutral, hydrophilic polymer coating that reduces binding of attachment proteins. The lid may seal the container by any suitable means. For example, the lid or cap 160 may comprise threads 165a that removably attach or releasably interlock with threads 165b on the housing 105. As an example, the lid may be a snap-on lid that releasably attaches to the housing (not shown). The container 100 may further comprise graduated or volumetric markings 170, such as markings 170 printed on an exterior surface 140 of sidewall 125 of the container 100.

In an aspect, a buffer as described herein prevents EVs from aggregating and sticking to one another during storage and handling. A buffered solution may be used in combination with coated storage containers as described herein for long-term storage and handling. In an embodiment, the buffer comprises a solution comprising an enzyme and a sugar to prevent EV aggregation. The enzyme helps to reduce any DNA or protein contaminant introduced into the spent media from the EV-producing cell, which can lead to aggregation and false quantification of genetic materials. The sugar acts as a cryo-protectant to slow the formation of ice crystals upon freezing. In solution, the enzyme and sugar allow for long-term EV storage and handling.

In an embodiment, the enzyme may comprise endonucleases, exonucleases, DNAses, RNases, strand-specific nucleases, Cas9 or other CRISPR associated protein nucleases, or combinations thereof. Nonlimiting examples of enzymes include DECONTAMINASE (a binuclease endonuclease available from AG Scientific Inc., San Diego, CA), recombinant Dr. Nuclease (a recombinant endonuclease available from Syd Labs, Boston, MA), PIERCE Universal Nuclease (a nuclease available from Thermo Fisher Scientific Inc., Waltham, MA), BENZALT (a genetically engineered non-specific endonuclease available from BioVision Inc., Milpitas, CA), CYANASE (a non-specific endonuclease available from Biophoretics, Sparks, NV), and BENZONASE (a genetically engineered endonuclease available from Merck KGaA, Darmstadt, Germany).

In an embodiment, the sugar may comprise cellobiose, chitobiose, isomaltose, kestose, lactose, lactulose, maltose, maltotriose, maltotriulose, mannobiose, melezitose, melibiose, nigerotriose, raffinose, sophrose, sucrose, trehalose, turanose, xylobiose, or combinations thereof.

Conventionally, EVs are typically used in the short-term. For example, EVs are typically used for research applications immediately after processing, such as in the exact same day as processing, due to degeneration of the EVs. In contrast, embodiments described herein allow for long-term storage and handling of EVs. In an embodiment, long-term EV storage may comprise up to 7 days. In an embodiment, long-term EV storage may comprise at least 7 days. In an embodiment, long-term EV storage may comprise from 7 days to 30 days. In an embodiment, long-term EV storage may comprise up to 30 days.

In an aspect, a method of storing extracellular vesicles (EVs) is provided. The method comprises collecting EVs in a storage container, such as a storage container described in embodiments herein. In an embodiment according to methods described herein, collecting EVs in a container further comprises collecting EVs post-diafiltration. The method further comprises adding a buffer to the container, and storing the EVs in the buffer. In embodiments, the buffer comprises a solution comprising an enzyme and a sugar.

In an embodiment according to methods described herein, storing the EVs in buffer comprises storage at temperatures in a range of about 25° C. to about −80° C. In an embodiment according to methods described herein, storing the EVs in buffer comprises storage for up to 7 days without degradation or decrease of EV concentration. In an embodiment according to methods described herein, storing the EVs in buffer comprises storage at 4° C. for up to 5 days.

In an embodiment, a freeze-thaw cycle comprises thawing the buffer comprising EVs from a frozen temperature to a room temperature. In an embodiment according to methods described herein, storing the EVs in buffer comprises storage over one or more freeze-thaw cycles. In an embodiment according to methods described herein, storing the EVs in buffer comprises storage for up to three freeze-thaw cycles.

### EXAMPLES

### Vero EV Stability During Storage in EV Purification Buffer

Vero EVs were stable up to 5 days at 4° C. (fridge) and three freeze-thaw cycles at −20° C. (freezer) in newly formulated EV Purification buffer (1L PBS+25 mM trehalose+20,000 U benzonase)

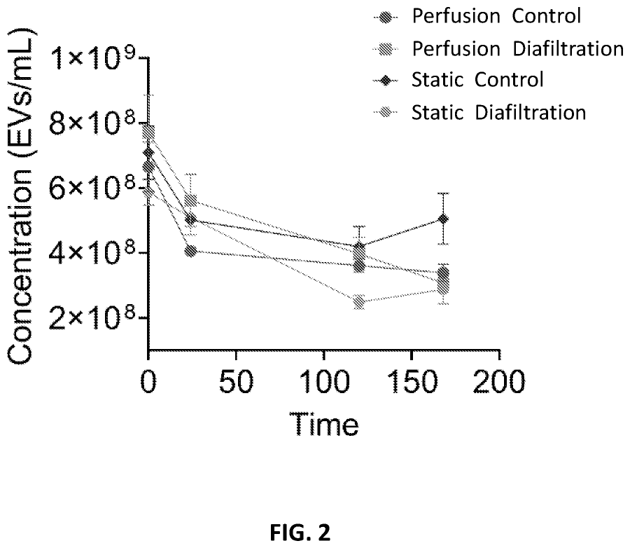

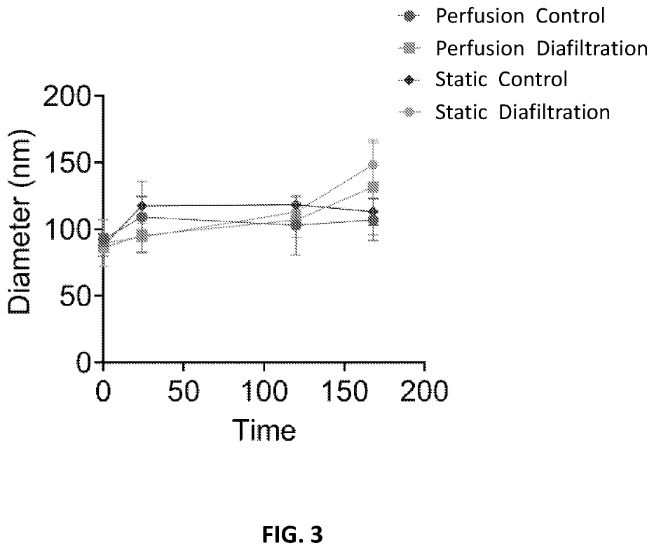

FIG. 2 and FIG. 3 depict the stability of Vero EVs post-diafiltration at 4° C. in EV purification buffer. Data shown mean±standard deviation, n=3 independent samples per group. FIG. 2 shows a graphical image depicting the concentration of EVs over time in embodiments of storage containers according to the present disclosure. FIG. 3 shows a graphical image depicting the diameter of EVs over time in embodiments of storage containers according to the present disclosure.

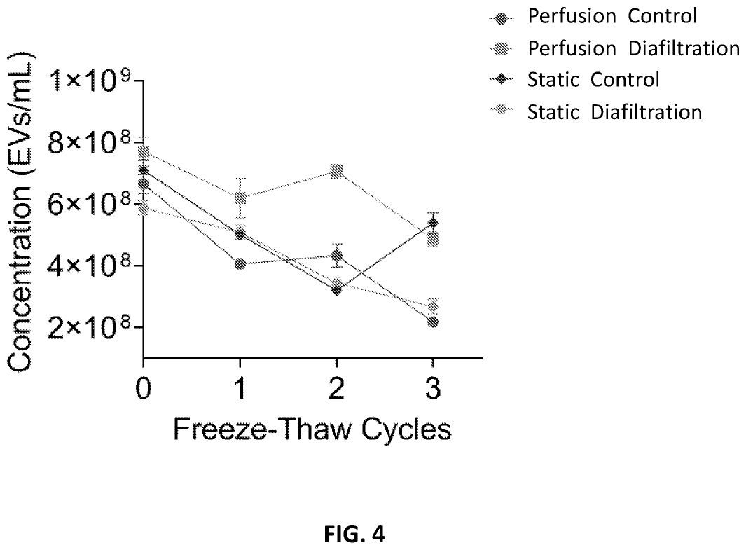

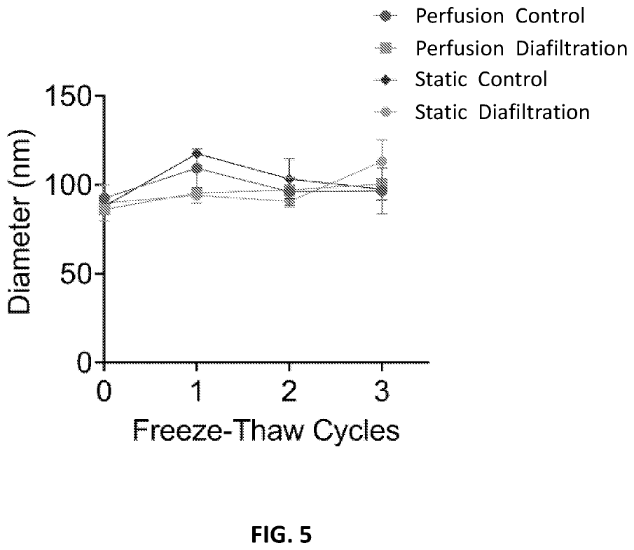

FIG. 3 and FIG. 4 depict the stability of Vero EVs post-diafiltration at −20° C. in EV purification buffer. Data shown mean±standard deviation, n=3 independent samples per group. FIG. 4 shows a graphical image depicting the concentration of EVs over freeze-thaw cycles in embodiments of storage containers according to the present disclosure. FIG. 5 shows a graphical image depicting the diameter of EVs over freeze-thaw cycles in embodiments of storage containers according to the present disclosure.

It will be appreciated that the various disclosed embodiments may involve particular features, elements or steps that are described in connection with that particular embodiment. It will also be appreciated that a particular feature, element or step, although described in relation to one particular embodiment, may be interchanged or combined with alternate embodiments in various non-illustrated combinations or permutations.

It is also to be understood that, as used herein the terms “the,” “a,” or “an,” mean “at least one,” and should not be limited to “only one” unless explicitly indicated to the contrary. Thus, for example, reference to “an opening” includes examples having two or more such “openings” unless the context clearly indicates otherwise.

Ranges can be expressed herein as from “about” one particular value, and/or to “about” another particular value. When such a range is expressed, examples include from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

All numerical values expressed herein are to be interpreted as including “about,” whether or not so stated, unless expressly indicated otherwise. It is further understood, however, that each numerical value recited is precisely contemplated as well, regardless of whether it is expressed as “about” that value. Thus, “a dimension less than 10 mm” and “a dimension less than about 10 mm” both include embodiments of “a dimension less than about 10 mm” as well as “a dimension less than 10 mm.”

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred.

While various features, elements or steps of particular embodiments may be disclosed using the transitional phrase “comprising,” it is to be understood that alternative embodiments, including those that may be described using the transitional phrases “consisting” or “consisting essentially of,” are implied. Thus, for example, implied alternative embodiments to a method comprising A+B+C include embodiments where a method consists of A+B+C, and embodiments where a method consists essentially of A+B+C.

Although multiple embodiments of the present disclosure have been illustrated in the accompanying Drawings and described in the foregoing Detailed Description, it should be understood that the disclosure is not limited to the disclosed embodiments, but is capable of numerous rearrangements, modifications and substitutions without departing from the disclosure as set forth and defined by the following claims.

1. A storage container for extracellular vesicles (EVs) comprising:
a container comprising:
a housing having an opening or aperture;
an interior volume defined within the housing; and
an interior surface of the housing comprising a neutral, hydrophilic polymer coating that reduces binding of attachment proteins; and
a lid or cap configured to removably attach to the housing to seal the opening or aperture of the container.

2. The storage container of claim 1, wherein the storage container is configured to store EVs up to 7 days.

3. (canceled)

4. The storage container of claim 1, wherein the storage container is configured to store EVs for 7 days to 30 days.

5. The storage container of claim 2, wherein the EV storage container is configured to store EVs without loss in yield.

6. The storage container of claim 2, wherein the EV storage container is configured to store EVs without sample contamination.

7. The storage container of claim 2, wherein the EV storage container is configured to store EVs at a temperature from 25° C. to −80° C.

8. The storage container of claim 2, wherein the storage container comprises a tube, a vial, or a flexible bag.

9. The storage container of claim 8, wherein the tube comprises a centrifugation tube, conical tube, or a culture tube.

10. (canceled)

11. The storage container of claim 5, wherein an interior surface of the lid or cap is configured to be in contact with the interior volume of the container when the lid or cap is attached to the housing.

12. The storage container of claim 11, wherein the interior surface of the lid or cap comprises a neutral, hydrophilic polymer coating that reduces binding of attachment proteins.

13. The storage container of claim 1, wherein the lid or cap comprises threads compatible with and configured to interlocking with threads on an exterior of the housing to seal the opening or aperture of the container.

14. The storage container of claim 1, wherein the lid or cap is configured to snap on an exterior of the housing to seal the opening or aperture of the container.

15. A method of storing extracellular vesicles (EVs) comprising:
collecting EVs in a storage container according to claim 1;
adding a buffer to the container, wherein the buffer comprises a solution comprising an enzyme and a sugar; and
storing the EVs in the buffer.

16. The method of claim 15, wherein storing the EVs in buffer comprises storage for 7 days to 30 days.

17. (canceled)

18. The method of claim 15, wherein storing the EVs in buffer comprises storage for up to 7 days.

19. (canceled)

20. The method of claim 15, wherein storing the EVs in buffer comprises storage for up to 7 days without loss in EV yield.

21. The method of claim 15, collecting EVs in a container further comprises collecting EVs post-diafiltration.

22. The method of claim 15, wherein storing the EVs in buffer comprises storage at 4° C. for up to 5 days.

23. The buffer of claim 15, wherein storing the EVs in buffer comprises storage over one or more freeze-thaw cycles.

24. (canceled)

25. The buffer of claim 23, wherein storing the EVs in buffer comprises storage for up to three freeze-thaw cycles.
